(19) United States
(12) Patent Application Publication
(10) Pub. No.: US 2004/0101861 A1
(43) Pub. Date: May 27, 2004

(54) Resonant cavity photodiode array for rapid DNA microarray readout

(76) Inventors: Roger G. Little, Bedford, MA (US); Kurt J. Linden, Wayland, MA (US)

Correspondence Address: NUTTER MCCLENNEN & FISH LLP, WORLD TRADE CENTER WEST, 155 SEAPORT BOULEVARD, BOSTON, MA, 02210-2604, US

(21) Appl. No.: 10/307,219

(22) Filed: Nov. 27, 2002

(51) Int. Cl. C12Q 1/68; C12M 1/34

(52) U.S. Cl. 435/6; 435/287.2

(57) ABSTRACT

The present invention provides a microarray having a plurality of micro-locations for confining selected photophores, for example, biological molecules exhibiting fluorescence spectra. The microarray can further include an array of optoelectronic photodetectors each of which is optically coupled with at least one of the micro-locations to detect radiation, for example, fluorescence radiation, that is emitted from the photophores confined in that micro-location. Each photodetector includes a resonant cavity that is formed of a front reflector and/or a back reflector having distributed Bragg reflector structures and a photo-detecting element disposed in the resonant cavity. The microarray can utilize either external optical excitation sources, such as lasers, LEDs, or can contain its own excitation sources in an integrated structure containing both optical radiation emitters, such as, vertical cavity surface emitting lasers or resonant cavity LEDs, and resonant cavity photodetectors. The integrated emitters and detectors can be either coaxially or adjacently located. Further, the microarray can include either separate sample array and excitation/detector array plates, or a single sample/excitation/detector array plate in which the photophore-containing sample molecules can be deposited directly on the excitation/detector array.

### BACKGROUND OF THE INVENTION

The present invention relates generally to microarrays, and more particularly, to improved microarrays that can be utilized for performing biological assays.

Microarrays of biological compounds are widely employed for biological analysis. For example, oligonucleotide probes, such as, known DNA fragments, can be immobilized on a substrate surface in the form of a regular array. A solution containing unknown target DNA sample, which has been labeled with a fluorophore, can then be applied to the substrate. Under certain conditions, hybridization can occur between target DNA sequences and the probes, if any, having complementary sequences. Subsequent rinsing of the substrate ensures that only those locations in the probe array in which hybridization occurred contain fluorophores. The excitation of the fluorophores at these locations by light having appropriate frequency, e.g., blue or ultraviolet radiation, can cause the fluorophores to fluoresce. The emitted fluorescence can then be analyzed to identify the specific sites where hybridization has occurred, thereby identifying the target sequences.

In general, the emission spectrum associated with each array location needs to be measured, and analyzed for peak wavelength, peak intensity, spectral linewidth and possibly spectral lineshape. Because microarrays typically include thousands of locations, performing such fluorescence measurement and analysis by traditional systems can be time-consuming and expensive.

Accordingly, there is a need for microarrays that allow rapid measurement and analysis of radiation originating from their micro-locations.

There is also a need for microarrays that allow rapid characterization of hybridization reactions occurring at their micro-locations.

### SUMMARY OF THE INVENTION

The present invention provides a microarray that includes a substrate having a plurality of micro-locations on a surface thereof, and an array of optoelectronic photo-detectors that are optically coupled to the micro-locations.

Each micro-location is capable of confining a photophore. The term “photophore,” as used herein, refers to any moiety, or a group of moieties, that can generate radiation, for example, a molecule that fluoresces, in response to a stimulating excitation, or a material that fluoresces without any excitation. A photophore can be one or a group of biological molecules, crystallites, or other substances containing fluorophores, and/or exhibiting bioluminescence, or any other material characterized by a specific optical emission spectrum. Further, a photophore may exhibit chemiluminescence or phosphorescence.

The optical coupling of the photodetectors to the micro-locations is such that each photodetector is optically coupled to at least one of the micro-locations in order to detect radiation originating from the photophores confined in that micro-location. In one embodiment, each photodetector can include a resonant cavity formed from a front reflector and/or a back reflector having distributed Bragg reflector (DBR) structures.

In a related aspect, each photodetector can include a photo-detecting element disposed in the reflector cavity for detecting radiation incident on the photodetector. The photodetector can further be designed for selective sensitivity to radiation of particular wavelengths within a desired range. In particular, the photo-detecting element can generate electrical signals in response to radiation transmitted through the front reflector (or directly from the micro-location surface, if no front reflector is utilized) into the cavity. Examples of suitable photo-detecting elements for use in the practice of the invention include, but are not limited to, photodiodes, PIN photodiodes, phototransistors, and avalanche photodiodes. The photo-detecting element is preferably sensitive to radiation having wavelengths that are substantially the same as one or more wavelengths of one or more modes of the resonant cavity formed by the front and/or the back reflectors. Further, each photodetector preferably exhibits a selected spectral response that at least partially overlaps with a frequency spectrum associated with radiation originating from the micro-location that is optically coupled to that photodetector, i.e., the radiation emitted by photophores confined in that micro-location.

In another aspect, the front reflector of a photodetector utilized in a microarray of the invention described above can serve as a filter, selectively transmitting radiation having one or more wavelength components in a wavelength range associated with radiation originating from the micro-location optically coupled thereto. The back reflector of the photodetector can be highly reflective to radiation in this wavelength range. For example, the reflectivity of the back reflector can be preferably at least 90% and, more preferably, at least 99%.

The substrates on which the micro-locations and the photodetectors are formed can be separate structures, or alternatively, they can form a unitary structure. Further, in some embodiments, a single substrate can be utilized for formation of the micro-locations as well as the photodetectors.

In another aspect, the photophores can be selected to be moieties of biological molecules, such as, fluorescence labels attached to oligonucleotides, peptides, or peptide nucleic acids, that exhibit fluorescence in response to an excitation, or bioluminescent materials. In such cases, each photodetector is designed to exhibit a spectral response that at least partially overlaps the fluorescence spectrum corresponding to radiation originating from the photophores confined in the micro-location that is optically coupled to that photodetector.

A microarray of the invention can be employed in a variety of applications. For example, in a method according to the teachings of the invention for processing radiation generated by a plurality of photophores, the photophores can be disposed on a plurality of micro-locations formed on a substrate surface, which are capable of confining the photophores. A plurality of optoelectronic photodetectors, having resonant cavity structures formed of a front reflector and/or a back reflector with distributed Bragg reflector structures, are then optically coupled to the micro-locations to detect radiation generated by photophores such that each photodetector detects radiation from one of the mircro-locations.

In another aspect, the invention provides a microarray that includes a substrate having a plurality of micro-locations that are capable of confining photophores, and further includes a plurality of optoelectronic emitters and photodetectors integrally formed in a single substrate optically coupled to the micro-locations in order to excite the confined photophores and detect radiation, such as, fluorescence, generated by the photophores in response to excitation. For example, the photo-emitters and the detectors can be formed in a single substrate as emitter/detector pairs optically coupled to the micro-locations such that the emitter of each pair can excite photophores in at least one of the micro-locations, and the detector of the pair can detect radiation emitted from these photophores in response to the excitation.

In a related aspect, the microarray devices of the present invention can have emitters and detectors integrally formed in a single substrate. For example, the micro-locations can be formed on a surface of the substrate in which the detectors and the emitters are formed. Alternatively, the micro-locations can be formed in a substrate separate from that employed for forming the emitter/detector array.

In another aspect, the emitters in the above microarray are formed, for example, by utilizing quantum well intermixing, so as to have specific emission spectra. Further, each emitter can be configured to emit light having a spectrum that at least partially overlaps with an absorption spectrum of photophores confined in a micro-location optically coupled to that emitter. In addition, each detector can have a spectral response function that at least partially overlaps with the spectrum of the radiation generated by photophores confined in a micro-location coupled to that detector in response to excitation by light generated by an emitter.

A variety of emitters and detectors can be employed for forming a microarray of the invention having an emitter/detector array integrally formed in a single substrate. For example, the emitters can be formed as distributed feedback diode lasers. And each photodetector can have a resonant cavity structure composed of a front reflector and a back reflector having distributed Bragg reflector structures, and a photo-detecting element disposed between the reflectors. Some suitable examples of photo-detecting elements include, but are not limited to, PIN photodiodes, phototransistors, or avalanche photo diodes.

Further understanding of the invention can be obtained by reference to the following detailed description in conjunction with the associated drawings briefly described below.

### BRIEF DESCRIPTION OF THE DRAWINGS

FIG. 1 schematically illustrates a microarray according to the teachings of the invention,

FIG. 2 schematically depicts the structure of an exemplary photodetector, having a resonant cavity, suitable for use in a microarray according to the teachings of the invention,

FIG. 3 is a schematic cross-sectional view of a microarray of the invention formed on a single substrate, and

FIG. 4 schematically depicts an exemplary application of a microarray of the invention for identifying unknown oligonucleotide target sequences,

FIG. 5A schematically illustrates another microarray according to the teachings of the invention having a substrate in which an array of emitter/detector pairs are integrally formed,

FIG. 5B is a cross-sectional view of the microarray of FIG. 5A,

FIG. 5C schematically illustrates the structure of a semiconductor light emitting device suitable for use in the emitter/detector array of FIG. 5A,

FIGS. 6A schematically illustrates a quantum well structure formed on a substrate between two barrier layers,

FIG. 6B schematically illustrate a quantum well intermixing technique for locally altering bandgap of the quantum well structure of FIG. 6A,

FIG. 7 schematically illustrates another microarray according to the teachings of the invention having a substrate that includes a plurality of emitter/detector pairs, each formed as an emitter disposed adjacent to a detector, and

FIG. 8 is a perspective view of another microarray of the invention in which a plurality of micro-locations, photo-emitters, and photodetectors are integrally formed in a single substrate.

### DETAILED DESCRIPTION

The present invention provides a microarray that includes a substrate on which a plurality of micro-locations are formed for confining selected photophores, for example, biological molecules exhibiting fluorescence spectra. The microarray can further include an array of optoelectronic photodetectors each of which is optically coupled with at least one of the micro-locations to detect radiation, for example, fluorescence radiation, that is emitted from the photophores confined in that micro-location. As described in more detail below, each photodetector includes a resonant cavity that is formed of a front reflector and/or a back reflector having distributed Bragg reflector structures, and a photo-detecting element disposed in the resonant cavity for detecting radiation.

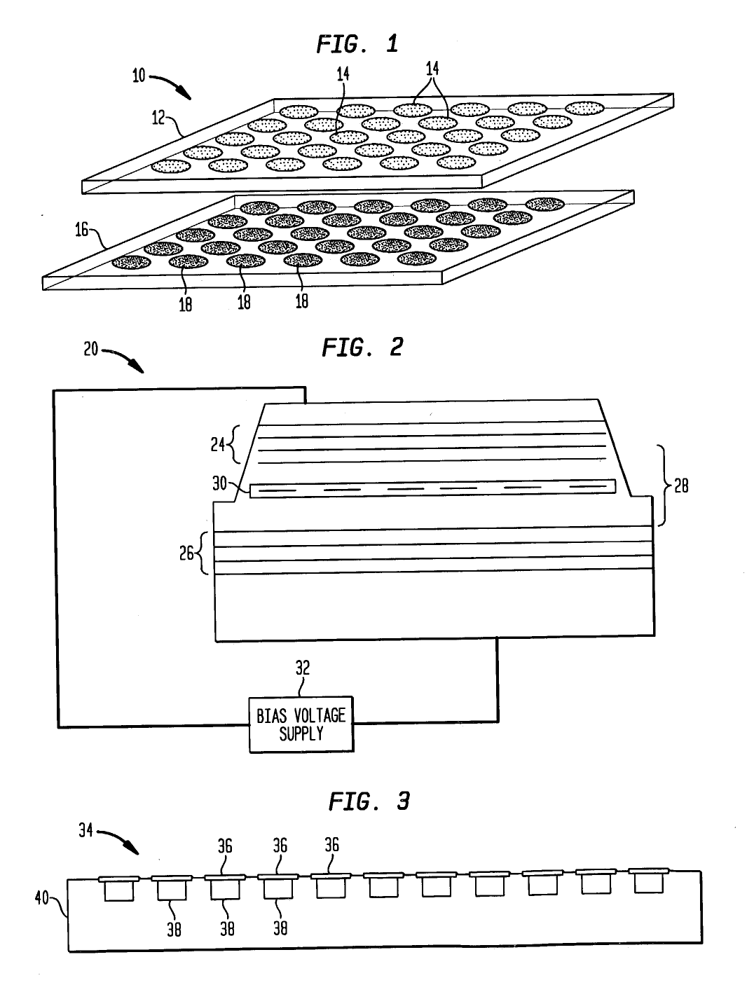

With reference to FIG. 1, an exemplary microarray 10 in accordance with the teachings of the invention can include a substrate 12 on which a plurality of micro-locations 14 are formed such that each micro-location can confine a selected photophore. Each photophore can emit radiation in response to an external excitation, such as radiation. For example, the photophore can be a biological molecule having a fluorophoric moiety, such as, a fluorescent-labled DNA molecule, that emits fluorescence radiation in response to excitation provided by a light source, such as, a laser or a light emitting diode (LED). The fluorescent label can be, for example, a fluorescent organic dye, such as, fluorescein or rhodamine dye, derivatized for attachment to the terminal 3′ or terminal 5′ of a DNA probe molecule via a linking moiety. The substrate 12 is preferably selected to be substantially transparent to the radiation emitted by the photophores to allow transmission of the emitted radiation to a plurality of photo-detectors optically coupled to the micro-locations, as discussed below. Some examples of suitable materials for forming the substrate 12 include, but are not limited to, glass, semiconductors, e.g., GaAs, InP, Si or any other group IV, group III-V, or other semiconductors. As described in detail below, a variety of known techniques can be utilized for forming the micro-locations 14 on the substrate 12. In an alternative configuration, the microlocations and the photophores are deposited on the bottom surface of the substrate 12 facing the substrate 16.

The microarray 10 further includes another substrate 16 in which a plurality of opto-electronic photodetectors 18 are formed. The photodetectors 18 are optically coupled to the micro-location 14 such that each photo-detector can detect radiation emitted from the photophores that are confined in at least one of the micro-locations 14. The optical coupling between the photodetectors 18 and the micro-locations 14 can be achieved, for example, by positioning the substrate 12 in proximity of the substrate 16 such that each of the photodetectors 18 is substantially in register with one of the micro-location 14 so as to subtend a solid angle spanned by at least a portion of the radiation originating from that micro-location. As described in detail below, each photodetector 18 can include a resonant cavity formed of a front reflector and/or a back reflector having distributed Bragg reflector structures, and can further include a photo-detecting element, such as, a PIN photodiode, that is disposed between these two reflectors for generating an electric signal in response to radiation incident thereon.

FIG. 2 schematically depicts an exemplary structure of a photodetector 20, for example, one of the photodetectors 18, that can be utilized in a microarray of the invention. The exemplary photodetector 20, which can be fabricated on a semiconductor substrate 22, e.g., a GaAs substrate, includes a front reflector 24 and a back reflector 26, each of which has a distributed Bragg reflector structure. The distributed Bragg reflector structures can be formed of alternating layers of dissimilar materials, such as, AlAs and GaAs, by utilizing known techniques that can include, but are not limited to, molecular beam epitaxy and/or chemical vapor deposition techniques.

In some embodiments, the front and the back reflectors 24 and 26 can define a resonant Fabry Perot etalon cavity 28, which can be formed, for example, of a semi-conductor material, such as, AlAs, GaAs or an alloy thereof, and can include a photo-detecting element, as discussed below. Although the resonant cavity 28 in general can exhibit a plurality of cavity modes, in many embodiments, the cavity 28 is relatively short, e.g., 1-2 microns, and hence would support only one longitudinal cavity mode. The cavity modes can be selected to coincide with one or more spectral components of the light emitted by the photophores.

The front reflector 24 can have a reflectivity at wavelengths corresponding to the modes of the resonant cavity 28 so as to allow at least partial transmission of spectral components at these frequencies into the resonant cavity, and also allow establishing standing waves in the cavity through multiple reflections of radiation from the front and the back reflectors. For example, the reflectivity of the front reflector 24 at selected wavelength regions can be in a range of about 20% to 95%. Further, the back reflector 26 can be constructed to be highly reflective over a range of wavelengths corresponding to the cavity modes. More specifically, the back reflector 28 is preferably at least about 95% reflective at these wavelengths. In this manner, the radiation emitted by the photophores confined at a micro-location optically coupled to the photodetector 20 will be trapped within the cavity 28. Alternatively, if the top detector region 24 is not a Bragg reflector, the fluorescent radiation entering the photodetector will be reflected by the back reflector 26 towards the optically active region 30, thereby enhancing the detection sensitivity.

A photo-detecting element 30 can be formed within the resonant cavity 28 to detect radiation incident thereon, i.e., to generate electrical signals in response to the radiation. The photo-detecting element is preferably responsive to radiation having spectral components that coincide with the reflectance windows of the Bragg reflector(s). A power supply 32 can apply a voltage to the photo-detecting element to bias the photo-detecting element into a desired operating range. A variety of photo-detecting elements can be employed to practice the present invention. For example, the photo-detecting element can be a PN junction, an avalanche photodiode, or a phototransistor. The photo-detecting element generates an electrical signal in response to the radiation having a selected spectral range and incident thereon. The electrical signals generated by the photodetector 20 can be transmitted, for example, to a computer (not shown) for analysis, as described in more detail below.

Further details regarding an exemplary photodetector having a resonant cavity structure that is suitable for use in the practice of the present invention can be found in U.S. Pat. No. 6,380,531 B1, entitled “Wavelength tunable narrow linewidth resonant cavity light detector” and herein incorporated by reference in its entirety.

Referring again to FIG. 1, the photodetectors 18 can be formed so as to detect radiation in a variety of different wavelength ranges. For example, the photodetectors can detect radiation having wavelength components in a range between about 450 nm to about 1700 nm for AlGaAs/GaAs semiconductor materials. Further, the photodetectors in a microarray of the invention can have the same or different spectral responses. For example, in some embodiments, the photodetectors in a microarray of the invention exhibit substantially similar spectral responses whereas in other embodiments, two or more of such photodetectors can exhibit dissimilar responses.

With continued reference to FIG. 1, a variety of techniques known in the art can be utilized to generate the micro-locations 14. For example, in some embodiments, an array of polymers having known monomer sequences, e.g., oligonucleotide sequences, are formed at a plurality of selected locations on a substrate, such as glass, to generate the array of micro-locations 14. Techniques for generating such monomeric arrays are known in the art. For example, U.S. Pat. No. 6,399,365, herein incorporated by reference, describes forming such micro-locations on a substrate by providing linker molecules whose terminal ends include reactive functional moieties having photo-removable protective groups. Photolithographic techniques can be employed to remove the protective groups from linker molecules in one or more selected portions of the substrate by selectively exposing those portions to light. The substrate is then contacted with a monomer that reacts with linker molecules whose protective groups have been removed. The monomer, which can be, for example, an oligonucleotide, also includes a photo-removable protective group. This allows selectively removing the protective groups of the monomers for reaction with a second monomer. In this manner, polymers of known sequences can be formed on selected locations of a substrate corresponding to the micro-locations. The polymers having known sequences can be a variety of biological molecules and can be utilized as probes in a variety of biological assays. Some examples of suitable biological molecules include, but are not limited to, oligonucleotides, peptides, and peptide nucleic acids.

Alternatively, the microlocations 14 can be generated by disposing agarose gel pads at pre-defined locations of the substrate 12. Each gel pad can provide a three-dimensional environment in which oligonucleotides of a known length and sequence and a test sample containing an unknown sequence can react. U.S. Pat. No. 5,851,772, herein incorporated by reference, provides further information regarding formation of microarray of biological molecules by utilizing agarose gel pads.

It should be understood that techniques for forming the micro-locations 14 are not limited to those described above. In fact, any technique that would allow confining selected photophores at a plurality of distinct locations on a substrate surface, which is substantially transparent to radiation emitted by the photophores, can be employed to practice the present invention. For example, a co-pending U.S. patent application entitled “Surface activated biochip,” filed on Apr. 11, 2002 and having a Ser. No. 10/120,974, and herein incorporated by reference, describes generating micro-locations on discrete regions of a substrate surface by subjecting these locations to an ion beam treatment.

Although the exemplary microarray 10 described above includes two separate substrates 12 and 16, one of which is utilized for forming the micro-locations 14 and the other is utilized for forming the array of photodetectors 18, in some other embodiments, the substrates 12 and 16 can form a unitary structure. This can be done by forming the microlocations, and depositing the photophores, directly on the semiconductor detector array structure. The detector's Bragg reflector(s) would be tuned to the photophores' fluorescence emission peak wavelength, which would be spectrally removed from the fluorescence excitation peak wavelength.

Such a unitary structure can include a single substrate that encompasses both the micro-locations and the photodetectors. For example, FIG. 3 schematically illustrates a cross-sectional view of another microarray 34 according to the teachings of the invention having a array of micro-locations 36 and an associated array of photo-detectors 38 formed on a single substrate 40. The substrate 40 can be, for example, a semiconductor, such as, GaAs, InP or other appropriate semiconductor material. Similar to the photodetectors 18 described above, the photodetectors 38 have resonant cavity structures formed by front and/or back reflectors having DBR structures, and a photo-detecting element that is disposed within this structure and can be configured to selectively detect radiation at frequencies that are substantially equal to the frequencies associated with one or more modes of the resonant cavity formed by the reflectors, or simply detect all wavelengths falling within the reflectance band of the Bragg mirror(s).

A microarray formed according to the teachings of the invention can find a variety of applications. For example, such a microarray can be utilized to generate a microarray of biological molecules, such as, DNA molecules, peptides or peptide nucleic acids, for performing a variety of biological assays. For example, microarrays of immobilized nucleic acid sequences can be employed in large scale hybridization assays in many genetic applications, such as mapping of genomes, monitoring of gene expression, DNA sequencing, genetic diagnosis, and genotyping of organisms. Such microarrays can also be utilized to identify optimal therapeutic protocols for early and rapid detection of a variety of disease states.

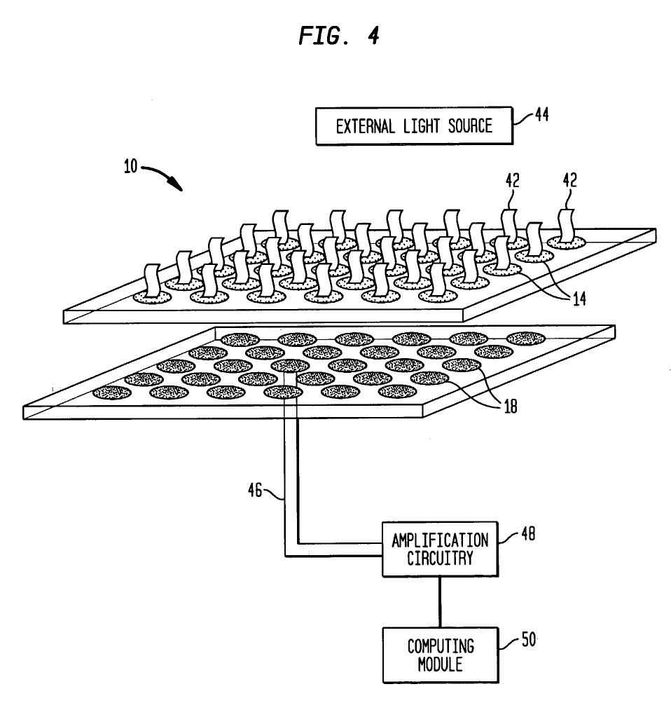

By way of example and with reference to FIG. 4, in an exemplary application of a microarray of the invention, such as, the exemplary microarray 10 described above, a plurality of oligonucleotide probes 42 with varying nucleotide sequences are deposited and confined, for example, by attachment to linking groups, to the micro-locations 14. The nucleotide probes can be prepared from naturally occurring nucleotides, or alternatively, can be synthesized by utilizing an automated nucleotide synthesizer, such as Applied Biosystems Instruments (ABI) synthesizer (U.S. Pat. Nos. 5,734,018 and 6,063,571). The oligonucleotide probes typically include a region of nucleotide sequence that can hybridize under stringent conditions to at least about 12 or 15, preferably about 20 or 25, and more preferably about 30, 35, 40, 45, 50, 55, 60, 65, or 75 consecutive nucleotides of a target nucleotide sequence. A test sample having an unknown target oligonucleotide sequence, which is labeled with a fluorophore, can then be applied to each of the micro-locations and incubated under conditions known in the art that allow hybridization of the target sequence with probes having complementary sequences. Following hybridization, the microarray can be washed in a manner known in the art so as to remove the unhybridized target sequences, and retain the hybridized sequences. So only those micro-locations having hybridized sequences contain flurophores that can be excited to emit radiation to be detected by the photodetector array 18.

With continued reference to FIG. 4, an external excitation source 44, such as a source that can provide blue or ultraviolet radiation (e.g., a laser or an LED), can be utilized to excite the fluorophore labels of the hybridized sequences, thereby eliciting fluorescence radiation from these flurophores. Each photodetector of the array 18 can detect fluorescence radiation, if any, from the micro-location to which it is optically coupled. An electrical signal generated by each of the photodetectors 18 in response to incident fluorescence is maximized when the fluorescence emission spectrum at least partially overlaps with the pre-designed resonant photodetector spectral response. The electrical signals generated by the photodetectors 18 can be transmitted via a conductive line 46 to an amplification circuitry 48 to amplify the signals. The amplified signals can then be transmitted to a computing module, such as, a computer or a dedicated processor, to be analyzed in order to generate a two-dimensional pattern of fluorescence intensity corresponding to the two-dimensional micro-locations 14.

Those having ordinary skill in the art will recognize that the teachings of the invention are not limited to two-dimensional microarrays, such as that described above. For example, a one-dimensional (line) microarray can be constructed in accordance with the teachings of the invention, as described above.

Thus, the microarray of the invention allows rapid processing of the fluorescence radiation by providing, for each micro-location, a dedicated photodetector, having a tailored spectral response, optically coupled to that micro-location. In addition, manufacturing of the microarrays of the invention are economically viable because established integrated circuit processing technology can be utilized for producing the arrays.

Some embodiments of the invention provide microarrays in which one or more light emitters for exciting photophores confined in a plurality of micro-locations and one or more photodetectors for detecting light emitted by the photophores in response to excitation are formed in the same substrate, as discussed in more detail below. Each emitter/detector pair can be associated with a micro-location so as to excite photophores confined in that micro-location, and further detect radiation, e.g., fluorescence, emitted by the photophores in response to exciation.

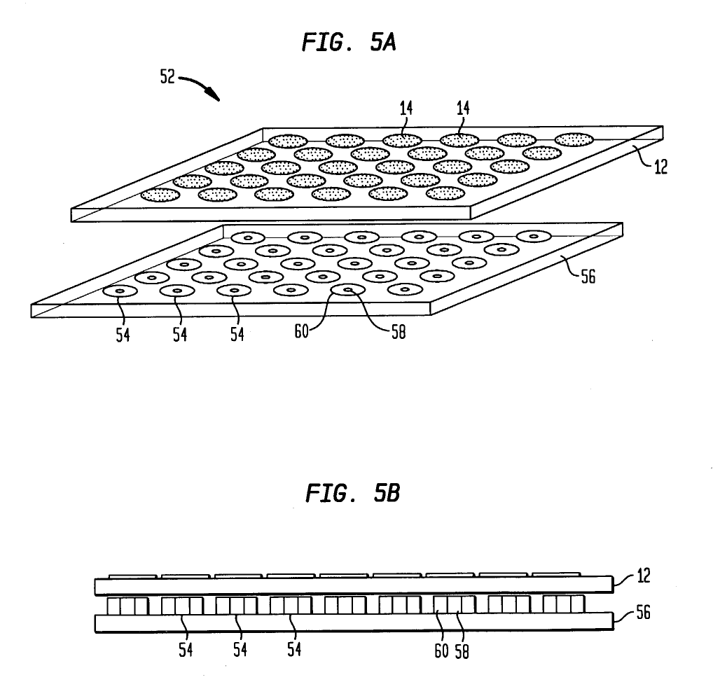

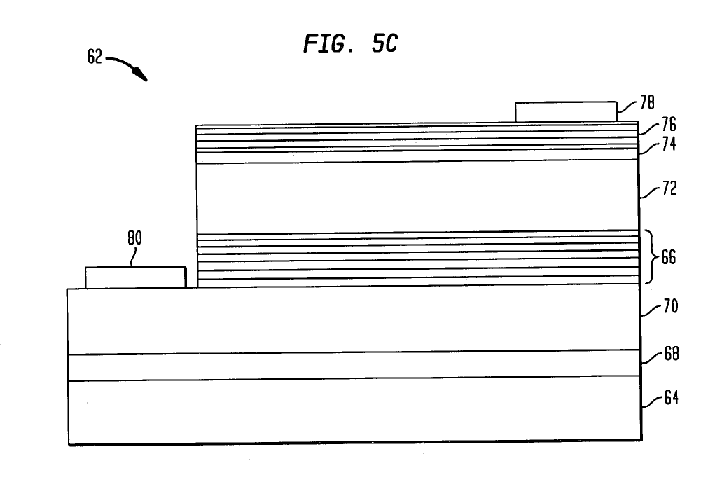

By way of example, FIGS. 5A and 5B schematically illustrate a microarray 52 according to the teachings of the invention in which a plurality of micro-locations 14 for confining selected photophores are formed on a substrate, e.g., glass or a semiconductor substrate, in a manner described above. The microarray 52 further includes an array 54 of light emitters and photodetectors integrally formed in another substrate 56 such that each emitter is coaxially surrounded by a photodetector. For example, a photo-emitter 58 is coaxially formed with a photodetector 60. The substrates 12 and 56 are positioned in proximity of each other, or in direct contact with another, such that each emitter/detector pair is optically coupled to at least one of the micro-locations 14. That is, the emitter of the pair can excite photophores confined in that micro-location, and the detector of the pair can detect any radiation, e.g., fluorescence, emitted by these photophores in response to the excitation.

The array 54 of emitter/detector pairs can be formed on the substrate 56, e.g., InP or silicon substrate, by utilizing, for example, photolithographical techniques known in the art. Further, the substrate 12 can be selected to allow transmission of light emitted by the emitters to the micro-locations, and further allow transmission of light originating from the micro-locations to the detectors. Alternatively, the photophores can be deposited on the underside of the microarray that faces the excitation emitter/detector array 56.

The detector in each emitter/detector pair can be, for example, a photodetector having a resonant cavity structure, such as those described above, that exhibits a narrow band response tailored to be, for example, in substantial resonance with at least a portion of the emission spectrum of photophores confined in a micro-location optically coupled to the emitter/detector pair.

A variety of emitters exhibiting different emission spectra can be utilized to form the above array of emitter/detector pairs. For example, the emitters can be semiconductor vertical cavity surface emitting lasers (VCSELs) or resonant cavity LEDs epitaxially formed on the substrate 56. Each emitter preferably has an emission wavelength that is tuned to the peak of optical absorption spectrum of the photophores confined in the micro-location optically coupled thereto.

By way of example, FIG. 5C schematically illustrates an exemplary VCSEL light emitting device 62 suitable for use as an emitter in the above emitter/detector array 54. The exemplary light emitting device 62, formed on a substrate 64 such as GaAs or Sapphire, includes a light emitting region 66 that is formed as a multiquantum well (MQW) structure of alternating quantum well and barrier layers. The device 62 further includes a buffer layer 68 deposited over the substrate 64, and an n-type semiconductor layer 70, e.g., AlInGaN, formed over the buffer layer 68. A p-type cladding layer 72 is formed over the emitting layer 66, and a layer 74, e.g., a GaN layer, is provided to decrease contact resistance with respect to transparent electrode layer 76. In the case of GaN-based devices, electrode pads 78 and 80 allow injecting current in the device to cause light emission. Further details regarding this type of light emitting device 62 can be found in U.S. Pat. No. 6,420,733 B2, herein incorporated by reference in its entirety.

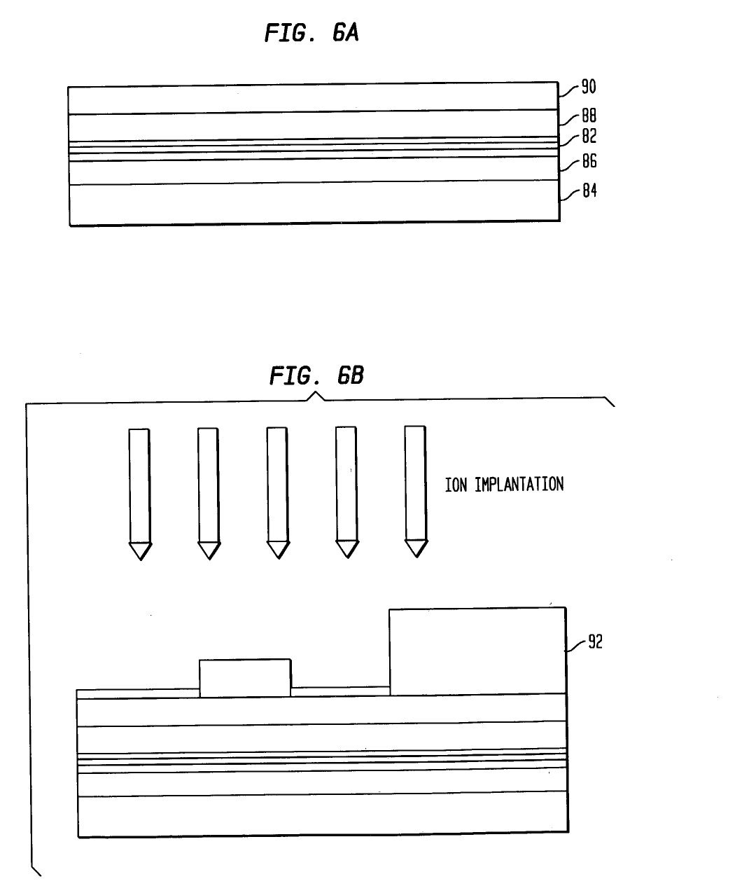

While in some embodiments of the invention the emitters exhibit substantially similar emission spectra, in some other embodiments the emitters can have different emission spectra, e.g., different emitters can emit at different wavelengths. In particular, quantum well intermixing (QWI) techniques can be utilized to generate an array of emitter/detector pairs, such as the above array 54, in which the emitters at different locations emit at different wavelengths. As known to those having ordinary skill in the art, a QWI technique allows modifying the energy bandgap of a grown quantum well by controlled lattice disordering without a need for epitaxial regrowth. For example, with reference to FIGS. 6A and 6B, a QWI method can locally modify the bandgap of a quantum well (or MQW) structure 82 formed on a substrate 84 between two barrier layers 86 and 88 and capped by a cladding layer 90, by an intial step of implanting neutral impurities, e.g., phosphorous ions, through a glass mask 92 having a selected thickness profile that varies from one location to another. The varying thickness of the glass mask controls the amount of phosphorous ions that are implanted in a region above the quantum well. A subsequent annealing process can then effect a gradation of the QW structure.

Further details regarding quantum well intermixing techniques can be found, for example, in U.S. Pat. No. 6,027,989 entitled “Bandgap tuning of semiconductor well structure,” herein incorporated by reference in its entirety.

Thus, referring again to FIGS. 5A and 5B, the emitters of the emitter/detector array 54 can be formed to emit light at different wavelengths. This advantageously allows utilizing, for example, different fluorphores exhibiting different absorption spectra at different locations.

The detector at each location can be formed to exhibit a response function that at least partially overlaps the fluorescence spectrum of the flurophores confined at the location optically coupled to that detector. While in some embodiments the spectral response functions of the detectors in the array 54 are similar, in some other embodiments detectors positioned at different locations can exhibit different response functions. For example, when the detectors include resonant cavity structures, the lengths of the resonant cavities can be modulated as a function of the detector's locations to provide different response functions.

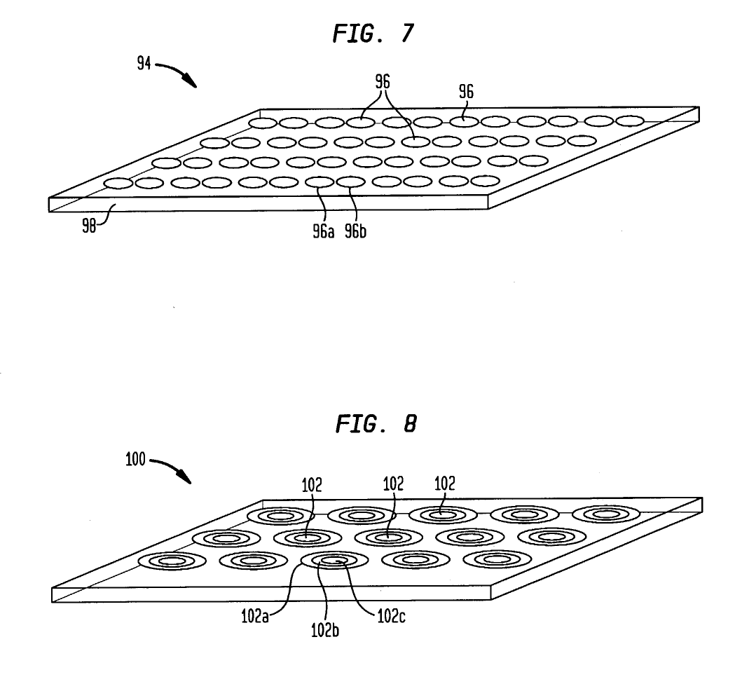

Although in the above microarray 52, each emitter/detector pair is formed as a coaxial structure with the detector surrounding the emitter, in another microarray 94 according to the teachings of the invention, shown in FIG. 7, an array of emitter/detector pairs 96 is formed in a substrate 98 by positioning each emitter adjacent to a detector. For example, emitter 96a is disposed in proximity of a detector 96b to form an emitter/detector pair. Each emitter/detector pair is optically coupled to one of the micro-locations (not shown), formed on a separate or the same substrate, that is capable of confining selected photophores, for example, in a manner described above.

In the microarrays 52 and 94 described above, the micro-locations and emitter/detector pairs are formed on separate substrates. FIG. 8 illustrates another microarray 100 according to the teachings of the invention in which a two-dimensional array 102 of micro-locations, emitter/detector pairs are integrally formed on a single substrate, e.g., a silicon substrate. At each micro-location, an emitter associated with that micro-location can excite photophores confined in that micro-location, and a detector associated with that micro-location can detect radiation generated by the excited photophores. For example, the micro-location 102a not only allows confining selected photophores, but it also includes an emitter 102b, and a detector 102c formed integrally in the same substrate for exciting the photophores confined in the micro-location 102a, and detecting any radiation generated by the excited photophores, respectively.

In some embodiments, one-dimensional excitation/detection arrays can be employed rather than two-dimensional arrays to minimize geometrical limitations associated with forming electrical interconnect patterns on high density two dimensional excitation/detection arrays. A two-dimensional sample array, e.g., an array of microlocations confining photophores, can be moved relative to the one-dimensional excitation/detection array, and readings can be taken line-by-line as the scanning of the two-dimensional microarray proceeds.

Those skilled in the art will appreciate that various modification can be made to the above embodiments without departing from the scope of the invention.

What is claimed is:

1. A microarray, comprising:
a substrate having a plurality of micro-locations on a surface thereof, each of said micro-locations being capable of confining a photophore, and
an array of optoelectronic photodetectors, each having a resonant cavity comprising at least a reflector having distributed Bragg reflector (DBR) structure, being optically coupled to said substrate surface such that each of said photodetectors is optically coupled to at least one of said micro-locations to detect radiation originating therefrom.

2. The microarray of claim 1, wherein said reflector is a front reflector.

3. The microarray of claim 2, wherein said resonant cavity further comprises a back reflector having a DBR structure.

4. The microarray of claim 1, wherein each of said photodetectors exhibits a selected spectral response that at least partially overlaps with a frequency spectrum associated with radiation originating from the micro-location optically coupled to said photodetector.

5. The microarray of claim 1, wherein each of said photodetectors further comprises a photo-detecting element disposed in said resonant cavity.

6. The microarray of claim 3, wherein each of said photodetectors further comprises a photo-detecting element disposed between said front and back reflectors.

7. The microarray of claim 5, wherein said photo-detecting element can be any of a PIN photodiode, a phototransistor, an avalanche photodiode or a photodiode.

8. The microarray of claim 1, wherein a surface of each photodetector optically coupled to one of said microlocations substantially transmits radiation having one or more frequency components in a frequency range associated with radiation originating from said optically coupled micro-location.

9. The microarray of claim 3, wherein the front reflector of said photodetector substantially transmits radiation having one or more frequency components in a frequency range associated with radiation originating from the micro-location optically coupled to said photodetector.

10. The microarray of claim 9, wherein the back reflector of said photodetector substantially reflects radiation having one or more frequency components in the frequency range associated with radiation originating from the micro-location optically coupled to said photodetector.

11. The microarray of claim 1, wherein at least one of said photodetectors comprises a resonant photodiode detector.

12. The microarray of claim 1, wherein said array of photodetectors is formed in the substrate having the micro-locations.

13. The microarray of claim 1, wherein said array of photodetectors is formed in a substrate separate from the substrate having the micro-locations.

14. The microarray of claim 1, wherein each of said photodetectors exhibits a spectral response in a range of about 450 nm to about 1700 nm.

15. The microarray of claim 1, wherein the substrate having the micro-locations comprises any of glass or semiconductor.

16. The microarray of claim 1, wherein said photophore comprises a biological molecule having a fluorescence emission spectrum.

17. The microarray of claim 16, wherein the photodetector optically coupled to the micro-location confining said biological molecule exhibits a spectral response that at least partially overlaps said fluorescence spectrum.

18. The microarray of claim 16, wherein said biological molecule can be any of oligonucleotides, peptides, or peptide nucleic acids.

19. The microarray of claim 1, further comprising an excitation source optically coupled to said substrate for eliciting radiation from said photophore.

20. The microarray of claim 19, wherein said excitation source comprises a light source.

21. The microarray of claim 20, wherein said light source comprises any of a laser or an LED.

22. A bioanalytical microarray, comprising
a substrate having a plurality of micro-locations on a surface thereof,
a plurality of biological molecules confined at said micro-locations, each biological molecule exhibiting a fluorescence spectrum, and
a plurality of optoelectronic photodetectors having resonant cavity structures comprising at least a reflector having distributed Bragg reflector structures and being optically coupled to said substrate such that each photodetector detects any of fluorescence and luminescence radiation originating from the molecules confined at one of said micro-locations.

23. The microarray of claim 22, wherein each of said photodetectors exhibits a spectral response function that at least partially overlaps any of the fluorescence and luminescence spectrum corresponding to radiation originating from molecules confined in one of said micro-locations and detected by said photodetector.

24. The microarray of claim 22, wherein said reflector is a front reflector.

25. The microarray of claim 24, wherein said resonant cavity further comprises a back reflector having a distributed Bragg reflector structure.

26. The microarray of claim 25, wherein each of said photodetectors comprises a photo-detecting element sandwiched between said front reflector and said back reflector.

27. The micro-array of claim 22, wherein each of said photodetectors comprises a resonant photodiode detector.

28. The method of claim 19, wherein said biological molecules can be any of oligonucleotides, peptides, or peptides nucleic acids.

29. A method of processing radiation generated by a plurality of photophores, the method comprising the steps of:
disposing photophores on a plurality of micro-locations formed on a substrate surface, each micro-location being capable of confining at least one photophore type, and
optically coupling a plurality of optoelectronic photodetectors having resonant cavity structures comprising a front reflector and a back reflector having distributed Bragg reflector structures to said substrate surface to detect radiation generated by said photophores such that each photodetector detects radiation originating from one of said micro-locations.

30. The method of claim 29, further comprising selecting at least one of said photodetectors to be formed as a photo-detecting element sandwiched between the front reflector and the back reflector.

31. The method of claim 29, further comprising selecting said photophores to comprise biological molecules.

32. The method of claim 31, further comprising selecting said biological molecules from the group consisting of oligonucleotides, peptides, and peptide nucleic acids.

33. A microarray, comprising:
a substrate having a plurality of micro-locations on a surface thereof, each of said micro-locations being capable of confining a photophore, and
a plurality of optoelectronic photodetectors and emitters integrally formed in a single substrate and arranged as a plurality of emitter/detector pairs optically coupled to said micro-locations such that for each pair the emitter of the pair emits light for exciting photophores confined in at least one of said micro-locations and the detector of the pair detects radiation generated by the confined photophores in response to said excitation.

34. The microarray of claim 33, wherein at least one of said photodetectors includes a resonant cavity comprising at least a reflector having a distributed Bragg reflector (DBR) structure.

35. The microarray of claim 34, wherein said reflector is a front reflector.

36. The microarray of claim 35, wherein said resonant cavity further comprises a back reflector having a DBR structure.

37. The microarray of claim 36, wherein said at least one photodetector further comprises a photo-detecting element disposed between said front and back reflectors.

38. The microarray of claim 33, wherein each of said photo-emitters emit light having a spectrum that at least partially overlaps an absorption spectrum of the photophores confined in a micro-location optically coupled thereto.

39. The microarray of claim 38, wherein each of said photodetectors exhibits a selected spectral response that at least partially overlaps with a spectrum associated with radiation originating from the micro-location optically coupled thereto.

40. The microarray of claim 33, wherein the emitters associated with at least two different pairs exhibit different emission spectra.

41. The microarray of claim 37, wherein said photo-detecting element can be any of a PIN photodiode, a phototransistor, an avalanche photodiode or a photodiode.

42. The microarray of claim 33, wherein said photodetectors comprise distributed feedback laser diodes.

43. The microarray of claim 33, wherein each emitter/detector pair is formed in a substrate as an emitter coaxially surrounded by a detector.

44. The microarray of claim 33, wherein each emitter/detector pair is formed in a substrate as an emitter disposed proximate to a detector.

45. The microarray of claim 33, wherein said plurality of photodetectors and emitters form a two-dimensional array.

46. The microarray of claim 33, wherein said plurality of photodetectors and emitters form a one-dimensional array.
